(19) United States
(12) Patent Application Publication
(10) Pub. No.: US 2021/0398632 A1
(43) Pub. Date: Dec. 23, 2021

(54) MEDICAL INFORMATION PROCESSING APPARATUS, MEDICAL INFORMATION PROCESSING METHOD, AND STORAGE MEDIUM

(71) Applicant: Canon Kabushiki Kaisha, Tokyo (JP)

(72) Inventor: Toru Kikuchi, Tokyo (JP)

(21) Appl. No.: 17/352,720

(22) Filed: Jun. 21, 2021

(30) Foreign Application Priority Data
Jun. 22, 2020 (JP) 2020-107226

(51) Int. Cl. G16H 15/00; G16H 30/40; G16H 50/70; G06T 7/00

(52) U.S. Cl. CPC G16H 15/00; G06T 7/0012; G16H 50/70; G16H 30/40

(57) ABSTRACT

A medical information processing apparatus comprises: a memory storing a program; and one or more processors which, by executing the program, cause the information processing apparatus to: obtain a plurality of findings for a case from a medical image as a target of a diagnosis; generate a description of a report based on at least some of the plurality of findings; and search a storage unit storing a plurality of cases for a specific case using a search condition including a designated finding designated as a condition to search for the specific case and discrimination information for discriminating whether the designated finding is a finding described in the report while discriminating whether the designated finding is a finding described in the report or a finding that is not described in the report.

### BACKGROUND OF THE INVENTION

### Field of the Invention

The present invention relates to a medical information processing apparatus, a medical information processing method, and a storage medium.

### Description of the Related Art

There is known a case search system that searches for similar cases using a result of analyzing a medical image by a computer or a document created by a doctor.

Japanese Patent Laid-Open No. 2012-198928 discloses a case search system that causes a user to select an attribute item and character information based on network information constituted by associating attribute items and information formed by character information of the attribute items with each other, thereby supporting input of a report, designating character information corresponding to an attribute item, and searching for a report.

Japanese Patent Laid-Open No. 2001-014326 discloses a search apparatus configured to accept input of a seed document and a search target structure as search conditions, extract a feature character string from the seed document, calculate the similarity between the combination of the extracted feature character string and the input structure and a combination of a feature character string of a document and a structure stored in a database, and present a document of high similarity as a search result.

In a document such as an interpretation report, a finding that a doctor has referred to in diagnosis is described. However, there can exist a case that is not described in the interpretation report but has a similar finding. For this reason, when searching for a similar case, it is preferable that not only a case whose finding is described in an interpretation report but also a case whose finding is not described in an interpretation report can be searched. It is also preferable that the search can be performed while discriminating the presence/absence of the description of a finding in an interpretation report. That is, it is preferable that only a case whose finding is described in an interpretation report can be searched, or only a case whose finding is not described in an interpretation report can be searched.

However, the method described in Japanese Patent Laid-Open No. 2012-198928 can search for a case only based on a finding described in an interpretation report and cannot search for a case that is not described in an interpretation report but has a similar finding. In addition, the method described in Japanese Patent Laid-Open No. 2001-014326 can designate, as a search condition, a structure in which a finding appears but cannot search for a case that is not described in an interpretation report but has a similar finding.

In consideration of the above-described problem, the present invention provides a medical information processing technique capable of searching for a case while discriminating whether a finding designated as a search condition is a finding described in a report or a finding that is not described in a report.

### SUMMARY OF THE INVENTION

According to one aspect of the present invention, there is provided a medical information processing apparatus comprising: a memory storing a program; and one or more processors which, by executing the program, cause the information processing apparatus to: obtain a plurality of findings for a case from a medical image as a target of a diagnosis; generate a description of a report based on at least some of the plurality of findings; and search a storage unit storing a plurality of cases for a specific case using a search condition including a designated finding designated as a condition to search for the specific case and discrimination information for discriminating whether the designated finding is a finding described in the report while discriminating whether the designated finding is a finding described in the report or a finding that is not described in the report.

Further features of the present invention will become apparent from the following description of exemplary embodiments (with reference to the attached drawings).

### BRIEF DESCRIPTION OF THE DRAWINGS

FIG. 1 is a view showing the configuration of a medical information processing system according to the first or second embodiment;

FIG. 2 is a view showing the hardware configuration of a medical information processing apparatus according to the first or second embodiment;

FIG. 3 is a view showing the functional configuration of the medical information processing apparatus according to the first or second embodiment;

FIG. 4 is a view showing an example of the user interface screen of the medical information processing apparatus according to the first embodiment;

FIG. 5 is a flowchart showing report generation processing of the medical information processing apparatus according to the first embodiment;

FIG. 6 is a flowchart showing search processing of the medical information processing apparatus according to the first or second embodiment;

FIG. 7 is a view showing an example of the user interface screen of the medical information processing apparatus according to the second embodiment; and

FIG. 8 is a flowchart showing report generation processing of the medical information processing apparatus according to the second embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made an invention that requires all such features, and multiple such features may be combined as appropriate. Furthermore, in the attached drawings, the same reference numerals are given to the same or similar configurations, and redundant description thereof is omitted.

### First Embodiment

In the first embodiment, a medical information processing apparatus that creates an interpretation report concerning a pulmonary nodule on a chest X-ray CT (Computed Tomography) image and searches for a specific case (a case and cases similar to the case) will be described.

The medical information processing apparatus according to this embodiment first estimates an imaging finding from an image of a pulmonary nodule. The imaging finding (to be also simply referred to as a “finding” hereinafter) is information representing the characteristic of the pulmonary nodule, such as “whole shape”, “border”, or “margin” of the pulmonary nodule.

An imaging finding concerning the “whole shape” takes a value such as “spherical”, “lobular”, “polygonal”, “wedge-shaped”, “flat”, or “irregular”. An imaging finding concerning the “border” takes a value such as “distinct” or “indistinct”, and an imaging finding concerning the “margin” takes a value such as “regular” or “irregular”. Hereinafter, “whole shape”, “border”, “margin”, and the like will be referred to as types of imaging findings, and “spherical”, “distinct”, “regular”, and the like will be referred to as values or contents of imaging findings. To estimate an imaging finding, a known CNN (Convolutional Neural Network) learned by supervised data can be used.

The medical information processing apparatus according to this embodiment estimates a diagnostic name using the estimated imaging finding. The diagnostic name is, for example, “benign pulmonary nodule”, “primary lung cancer”, or “metastatic lung cancer”. To estimate the diagnostic name, a known Byesian Network learned by supervised data can be used. Also, when estimating the diagnostic name, the medical information processing apparatus calculates the influence degree of each input imaging finding for the estimated diagnostic name. The medical information processing apparatus selects an imaging finding whose influence degree exceeds a predetermined value, and automatically generates a report (to be also referred to as an “interpretation report” hereinafter) by applying the selected imaging finding and the diagnostic name to a template. Also, the medical information processing apparatus accumulates the estimated imaging finding, the diagnostic name, and the automatically generated report as records in tables in a known RDB (Relational Database).

(Configuration of Medical Information Processing System)

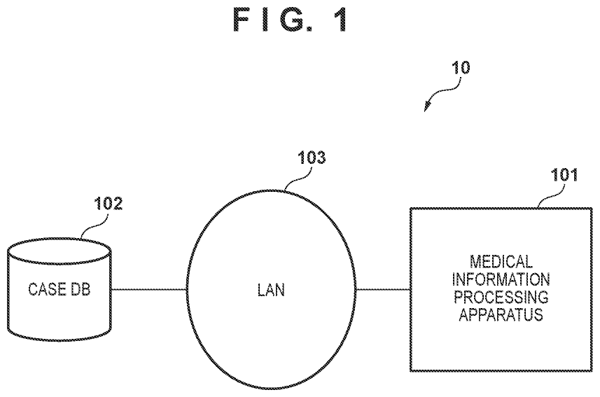

FIG. 1 is a view showing the configuration of a medical information processing system 10 including the medical information processing apparatus according to this embodiment. As shown in FIG. 1, the medical information processing system 10 includes a case database (to be referred to as a “case DB” hereinafter) 102, a medical information processing apparatus 101, and a LAN (Local Area Network) 103.

The case DB 102 functions as a storage unit that stores a medical image captured by an apparatus such as an X-ray CT apparatus for capturing a medical image, medical information accompanying the medical image, and a plurality of cases. The case DB 102 also provides a database function of searching for a medical image using medical information and obtaining a medical image via the LAN 103. For the database function, a known RDB can be used.

(Hardware Configuration)

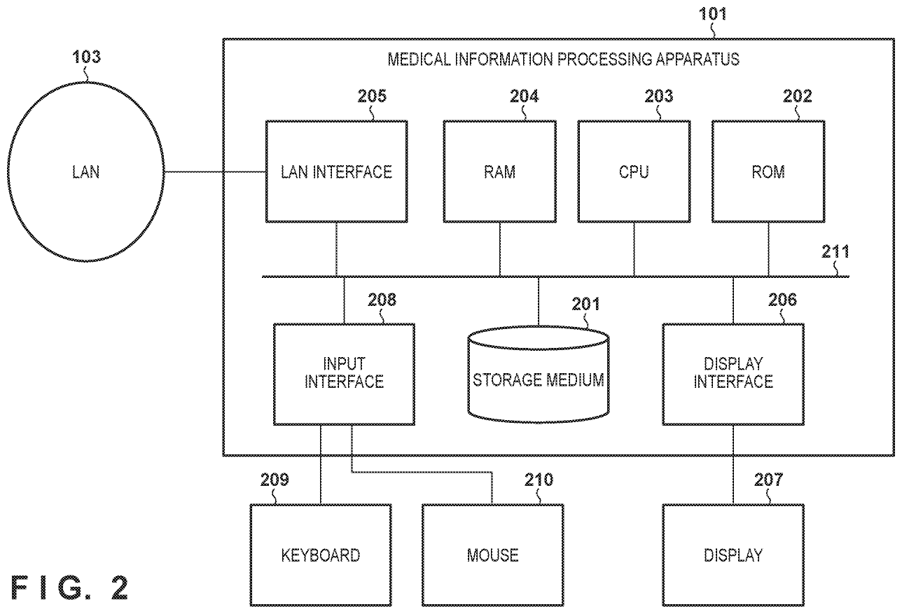

FIG. 2 is a view showing the hardware configuration of the medical information processing apparatus 101 according to this embodiment. Referring to FIG. 2, the medical information processing apparatus 101 includes a storage medium 201, a ROM (Read Only Memory) 202, a CPU (Central Processing Unit) 203, and a RAM (Random Access Memory) 204. The medical information processing apparatus 101 further includes a LAN interface 205, an input interface 208, a display interface 206, and an internal bus 211.

The storage medium 201 is a storage medium such as an HDD (Hard Disk Drive) that stores an OS (Operating System), processing programs configured to perform various kinds of processing according to this embodiment, and various kinds of information. The ROM 202 stores programs such as a BIOS (Basic Input Output System) configured to initialize the hardware and activate the OS. The CPU 203 performs arithmetic processing when executing the BIOS, the OS, or a processing program. The RAM 204 temporarily stores information when the CPU 203 executes a program. The LAN interface 205 is an interface corresponding to a standard such as IEEE (Institute of Electrical and Electronics Engineers) 802.3ab and configured to perform communication via the LAN 103.

A display 207 displays a user interface screen, and the display interface 206 converts screen information to be displayed on the display 207 into a signal and outputs it to the display 207. The CPU 203 and the display interface 206 function as a display control unit that controls display on the display 207. A keyboard 209 performs key input. A mouse 210 designates a coordinate position on a screen and inputs a button operation. The input interface 208 receives signals from the keyboard 209 and the mouse 210. The internal bus 211 transmits signals when communication is performed between the blocks.

(Functional Configuration)

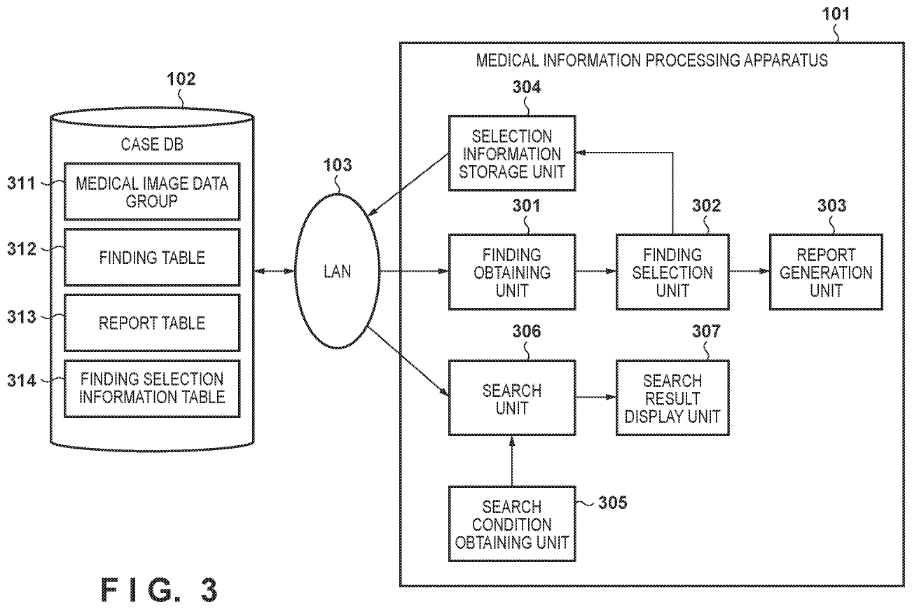

FIG. 3 is a view showing the functional configuration of the medical information processing apparatus 101 according to this embodiment. In the functional configuration shown in FIG. 3, the medical information processing apparatus 101 includes a finding obtaining unit 301, a finding selection unit 302, a report generation unit 303, a selection information storage unit 304, a search condition obtaining unit 305, a search unit 306, and a search result display unit 307. These functional blocks can be implemented when the CPU 203 functioning as the control unit of the medical information processing apparatus 101 executes a processing program read out from the storage medium 201. Each functional block may be formed by an integrated circuit or the like if it provides the same function.

The case DB 102 stores a medical image data group 311, a finding table 312, a report table 313, and a finding selection information table 314. The medical image data group 311 stores medical image data of a plurality of cases. Each of the finding table 312, the report table 313, and the finding selection information table 314 is a table of an RDB, and stores the information of a plurality of cases while setting one case as one record. Here, finding selection information is information for specifying a finding used for the description of a report. Note that a finding used for the description of a report will also be referred to as a finding described in a report hereinafter.

The finding obtaining unit 301 obtains a plurality of imaging findings for a case from a medical image as a diagnosis target. The finding obtaining unit 301 obtains a medical image as a diagnosis target and analyzes the medical image, thereby obtaining a plurality of findings. More specifically, the finding obtaining unit 301 obtains a medical image as a diagnosis target from the medical image data group 311, and estimates a plurality of imaging findings for a lesion from the obtained medical image using a CNN. The estimated imaging findings are the values of imaging findings of a plurality of types. One imaging finding may be estimated by one CNN, or a plurality of imaging findings may be estimated by one CNN. The finding obtaining unit 301 stores the obtained imaging findings in the finding table 312.

The finding selection unit 302 (first finding selection unit) selects, from the plurality of imaging findings obtained by the finding obtaining unit 301, some of the plurality of imaging findings as imaging findings to be used to generate a report. In imaging finding selection, first, a diagnostic name is estimated from the imaging findings. Next, the finding selection unit 302 calculates the influence degree of each imaging finding for the estimated diagnostic name. Finally, the finding selection unit 302 selects an imaging finding whose calculated influence degree is a predetermined value or more as an imaging finding to be used to generate a report. Here, the finding selection unit 302 can use a Byesian Network to estimate the diagnostic name. Here, the influence degree is the difference between the likelihood of the diagnostic name estimation result in a case in which an imaging finding is not input and the likelihood of the diagnostic name estimation result in a case in which an imaging finding is individually input.

The report generation unit 303 generates the description of a report concerning a case based on at least some of the plurality of selected findings. The report generation unit 303 generates the description of a report sentence by applying the imaging findings selected by the finding selection unit 302 to a template set in advance. For example, when findings {X}=“irregular” and {Y}=“spiculation” and “serrated margin” are applied to a template “{X} nodule is seen in the right lung, which includes {Y}”, the report generation unit 303 generates a report sentence “irregular nodule is seen in the right lung, which includes spiculation and serrated margin”. The report generation unit 303 stores the generated report in the report table 313. Note that the template and the imaging findings described above are merely examples and are not limited to these.

The selection information storage unit 304 stores specification information for specifying the finding used to describe the report in the finding selection information table 314 in the case DB 102 (storage unit). That is, the selection information storage unit 304 stores, in the finding selection information table 314, selection information (to be also referred to as “specification information”) that is information used to specify the imaging findings selected by the finding selection unit 302. The selection information is information that provides, for each of the imaging findings, a flag (information “1” or “0”) representing whether it is a selected imaging finding or not.

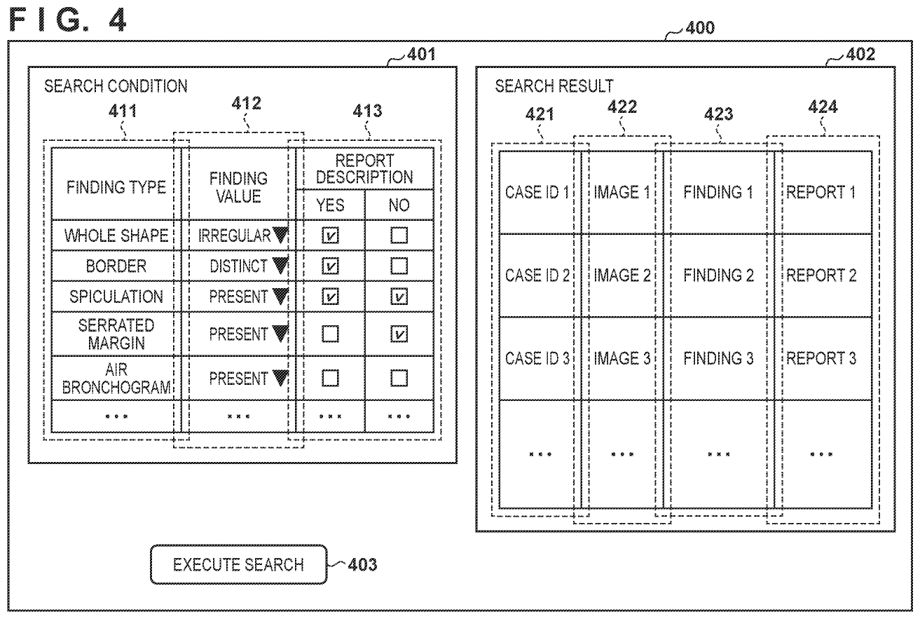

The search condition obtaining unit 305 obtains search condition information. The search condition obtaining unit 305 obtains a search condition including a designated finding designated as a search condition (to be also referred to as “search condition information” hereinafter) for searching for a specific case (a case and cases similar to the case) and discrimination information for discriminating whether the designated finding is a finding described in a report (that is, whether the designated finding was used to generate a report). Here, the search condition information includes the type and value of an imaging finding (designated finding) serving as a key when searching for a specific case (a case and cases similar to the case), and a flag (discrimination information) representing whether the imaging finding was used for a report. The search condition obtaining unit 305 obtains the search condition information via a user interface screen to be described with reference to FIG. 4.

The search unit 306 searches for a specific case (a case and cases similar to the case) using the finding table 312 and the finding selection information table 314 in accordance with the search condition information obtained by the search condition obtaining unit 305. Based on the discrimination information and the selection information (specification information) that is information for specifying an imaging finding, the search unit 306 searches the case DB 102 (storage unit) for a specific case (a case and cases similar to the case) while discriminating whether the imaging finding (designated finding) serving as a key in the search is a finding described in a report or a finding that is not described in a report. More specifically, in addition to the type and value of an imaging finding serving as a key when searching for a specific case (a case and cases similar to the case), the search unit 306 discriminates whether the imaging finding was used for a report and performs a search using both a case whose finding was used for a report and a case whose finding was not used for a report. The search unit 306 performs the search using the RDB when executing search processing.

Note that in this embodiment, an example in which a similar case is searched will be described below as the search of a specific case (a case and cases similar to the case). However, the present invention is not limited to the search of a similar case and can be similarly applied to a general case search.

That is, using the search condition including the designated finding designated as a condition to search for a specific case (a case and cases similar to the case) and discrimination information for discriminating whether the designated finding is a finding described in a report, the search unit 306 discriminates whether the designated finding is a finding described in a report or a finding that is not described in a report, and searches the storage unit (case DB 102) storing a plurality of cases for the specific case. Here, the storage unit (case DB 102) stores specification information for specifying a finding used to describe a report for every plurality of cases, and the search unit 306 searches for a specific case (a case and cases similar to the case) based on the specification information and the discrimination information.

The search result display unit 307 displays the search result by the search unit 306 on the display 207 (display unit) together with the search condition information obtained by the search condition obtaining unit 305. The search result display unit 307 performs display control to display the search result by the search unit 306 and the search condition information obtained by the search condition obtaining unit 305 in a predetermined value region on a user interface screen to be described with reference to FIG. 4.

(User Interface Screen)

FIG. 4 is a view showing an example of a user interface screen 400 of the medical information processing apparatus 101 according to this embodiment. The user interface screen 400 is displayed on the display 207, and an operation for the user interface screen 400 is performed by the keyboard 209 or the mouse 210. The CPU 203 and the display interface 206 function as a display control unit and control display on the display 207.

Referring to FIG. 4, the user interface screen 400 includes a search condition designation region 401, a search result display region 402, and a search execution button 403.

The search condition designation region 401 is a region used to designate a search condition and is formed by a finding type display region 411, a finding value designation region 412, and a report description designation region 413.

Types of imaging findings are displayed in the finding type display region 411. Here, the types of imaging findings include “whole shape”, “border”, “spiculation”, “serrated margin”, “air bronchogram”, and the like.

In the finding value designation region 412, the values of imaging findings corresponding to the finding types displayed in the finding type display region 411 are designated. Each row of the finding value designation region 412 is displayed as a pull-down menu. For example, when an icon (black triangle) on the right side of a finding value displayed as “irregular” is clicked by the mouse 210, a list of finding values (values) of the imaging finding, such as “spherical”, “lobular”, “polygonal”, “wedge-shaped”, “flat”, and “irregular”, which the finding type “whole shape” can take, is displayed as a candidate display. When a finding value (value) of the imaging finding in the candidate display is selected by clicking of the mouse 210, the clicked finding value (value) of the imaging finding can be designated.

In the report description designation region 413, whether the imaging finding of each finding type displayed in the finding type display region 411 is a finding described in a report or a finding that is not described in a report is designated. Each row of the report description designation region 413 includes two check boxes. When a check box of a column “present” is checked, concerning the corresponding finding, the search unit 306 searches for a case described in a report. When a check box of a column “absent” is checked, the search unit 306 searches for a case that is not described in a report. When both “present” and “absent” are checked, a case described in a report and a case that is not described in a report are searched. In the example shown in FIG. 4, cases that satisfy search conditions that “whole shape” is “irregular”, “border” is “distinct” (described in a report), “spiculation” is “present” (a description in a report does not matter), and “serrated margin” is “present” (not described in a report) are searched.

The search result display region 402 is a region used to display a search result, and includes a case ID display region 421, an image display region 422, a finding display region 423, and a report display region 424. In the case ID display region 421, pieces of identification information (identifiers) for uniquely identifying found cases (similar cases) are displayed.

In the image display region 422, medical images of found cases are displayed. The search result display unit 307 displays information for identifying a similar case searched by the search unit 306 in the case ID display region 421 of the display 207, and displays a medical image representing the similar case in the image display region 422 of the display 207. The medical image is a medical image obtained from the medical image data group 311. The finding obtaining unit 301 obtains a plurality of medical images from the medical image data group 311. If a medical image is formed by a plurality of tomographic images, an image representing a case designated by the user in advance is displayed.

In the finding display region 423, imaging findings of found cases are displayed. The imaging findings are imaging findings obtained from the finding table 312. In the report display region 424, reports of found cases are displayed. The reports are reports obtained from the report table 313.

The search result display unit 307 displays the search result while discriminating whether each of findings (finding 1, finding 2, finding 3, . . . ) in the finding display region 423, which are searched based on designated findings, is a finding described in a report or a finding that is not described in a report.

The search execution button 403 is a button used to instruct execution of a search according to search conditions designated in the search condition designation region 401. When the search execution button 403 is clicked by the mouse 210, a search is executed, and the result of the executed search is displayed in the search result display region 402.

(Report Generation Processing Procedure)

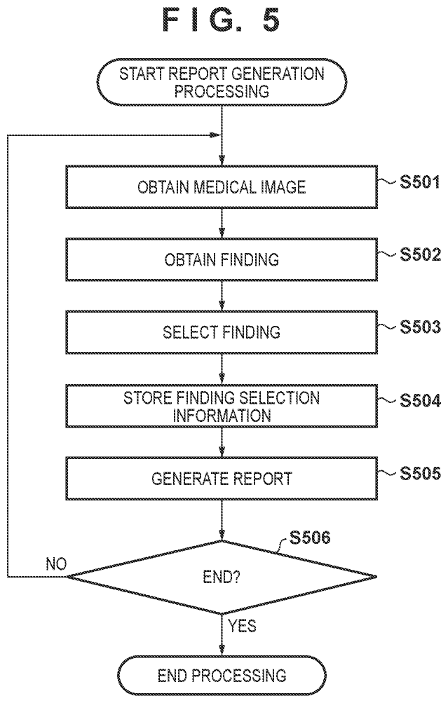

FIG. 5 is a flowchart showing the procedure of report generation processing of the medical information processing apparatus 101 according to this embodiment. Report generation processing is started based on an instruction by another apparatus included in the medical information processing system 10, or another system, or a user after activation of the medical information processing apparatus 101. When starting the processing, a case as the target of processing is designated.

In step S501, the finding obtaining unit 301 obtains medical images of the target case from the medical image data group 311 via the LAN 103 (network).

In step S502, the finding obtaining unit 301 estimates imaging findings from the medical images obtained in step S501 and stores the estimated findings in the finding table 312.

In step S503, the finding selection unit 302 selects imaging findings to be used to generate a report from the imaging findings obtained in step S502.

In step S504, based on the imaging findings selected in step S503, the selection information storage unit 304 stores, in the finding selection information table 314, finding selection information for specifying a finding described in a report (an imaging finding used to generate a report).

In step S505, the report generation unit 303 generates a report sentence using the imaging findings selected in step S503, and stores the generated report sentence in the report table 313.

In step S506, the OS determines the presence/absence of an end of the processing. If an end is detected (YES in step S506), the processing is ended. If an end is not detected (NO in step S506), the process returns to step S501 to repeat the same processing.

(Search Processing Procedure)

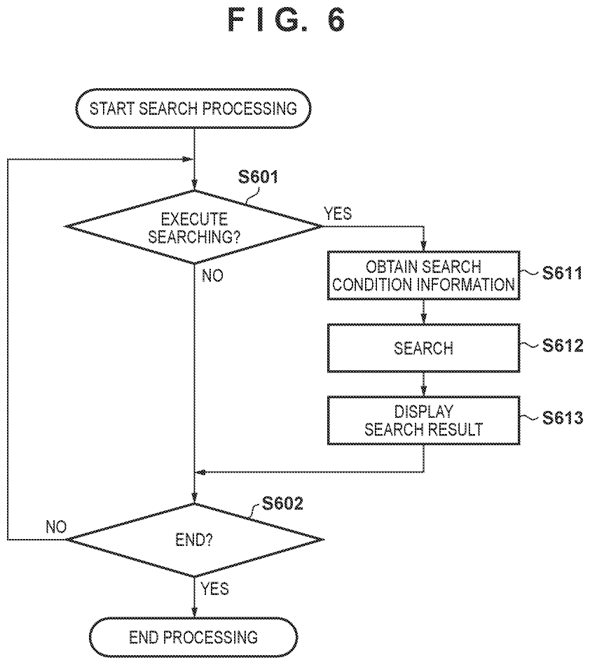

FIG. 6 is a flowchart showing the procedure of search processing of the medical information processing apparatus 101 according to this embodiment. Search processing is started based on an instruction by another apparatus included in the medical information processing system 10, or another system, or a user after activation of the medical information processing apparatus 101.

In step S601, the search unit 306 determines whether execution of a search is instructed. Execution of a search is instructed by clicking the search execution button 403 by the mouse 210. If execution of a search is instructed (YES in step S601), the process advances to step S611. If execution of a search is not instructed (NO in step S601), the process advances to step S602.

In step S602, the OS determines the presence/absence of an end of the processing. If an end is detected (YES in step S602), the processing is ended. If an end is not detected (NO in step S602), the process returns to step S601 to repeat the same processing.

In step S611, the search condition obtaining unit 305 obtains search condition information. The search condition information is information (discrimination information) for designating the type and value of an imaging finding serving as a key of the search and whether the imaging finding is described in a report. The search condition obtaining unit 305 obtains the search condition information via the search condition designation region 401 of the user interface screen shown in FIG. 4.

In step S612, the search unit 306 searches the case DB 102 for a case based on the search condition information obtained in step S611. In accordance with the search condition information obtained by the search condition obtaining unit 305, the search unit 306 searches for a similar case using the finding table 312 and the finding selection information table 314 in the case DB 102.

In step S613, the search result display unit 307 displays the result of the search in step S612 in the search result display region 402. Here, the search result display unit 307 displays the search result by the search unit 306 in the search result display region 402 together with the search condition information obtained by the search condition obtaining unit 305.

As described above, according to this embodiment, when searching for a case based on an imaging finding, search processing is executed by designating an imaging finding as a search condition and also designating whether the imaging finding is described in a report or not. This makes it possible to search for a case while allowing the user to discriminate an imaging finding described in a report and an imaging finding that is not described.

### Modification of First Embodiment

The medical information processing apparatus 101 may set an image of a part other than chest, such as abdomen, mammary gland, or head, to the target. In addition, a medical image captured by an apparatus other than X-ray CT, such as MRI (Magnetic Resonance Imaging), ultrasonic wave, or simple X-ray, may be set to the target. The lesion is not limited to a pulmonary nodule, and a lesion other than a pulmonary nodule, such as a diffuse pulmonary disease, breast mass, or hepatic mass, may be set to the target. Also, a diagnosis other than an image diagnosis, such as a pathologic diagnosis or a clinical diagnosis, may be set to the target. In this case, not an imaging finding but a finding according to the target diagnosis such as a clinical diagnosis such as visual inspection, palpation, or blood test or a pathologic diagnosis can be set to the target. A report to be generated may be text information used in a document other than an interpretation report, such as a pathologic diagnosis report or a medical record.

The finding obtaining unit 301 may obtain a finding not by a CNN but by a method using another machine learning for, for example, extracting an image feature amount and performing estimation by an SVM (Support Vector Machine). The finding may be obtained from another apparatus such as an image processing workstation. The finding may be input by the user via the user interface screen. Alternatively, the finding may be extracted from text information such as a natural sentence.

The finding selection unit 302 may infer the diagnostic name using a DNN (Deep Neural Network), calculate the gradient of each input node for the inference result, and select an imaging finding to be used to generate a report using the magnitude of the gradient as an influence degree. A supervised data that uses all findings as an input and a selected finding as an output may be created, and an imaging finding may be selected by a selector that has machine-learned the supervised data. Alternatively, a rule base that uses all findings as an input and a selected finding as an output may be constructed, and an imaging finding may be selected by the rule base.

The report generation unit 303 may generate the description of a report by sentence generation using a known Markov chain, an LSTM (Long Short-Term Memory), or the like.

The selection information storage unit 304 may store finding selection information as an attribute item of the finding table 312. Also, the information may be stored in a device or storage medium different from the case DB 102.

The search condition obtaining unit 305 may obtain the presence/absence of a description in a report from a setting file or setting data. The search condition obtaining unit 305 may obtain the presence/absence of a description in a report by voice input. Also, in accordance with a setting file or setting data, the search condition obtaining unit 305 may obtain one of the presence and absence of a description in a report by applying it to all imaging findings.

The search result display unit 307 may display a case for which the value of an imaging finding is the same as a search condition, but the condition concerning the presence/absence of a description in a report is different as reference information together with the search result. Also, the search result display unit 307 may control display in the search result display region 402 by displaying each imaging finding in a different mode (for example, a mode discriminated by changing at least one of the display color and the display size) based on the presence/absence of a description in a report.

### Second Embodiment

A medical information processing apparatus 101 according to the second embodiment is configured by adding a function of causing a user to designate an imaging finding to be used to generate a report to the medical information processing apparatus 101 according to the first embodiment. In addition, the medical information processing apparatus 101 according to this embodiment discriminatively searches for a finding automatically described in a report, a finding manually described in a report based on a designation by the user, and a finding that is not described in a report. Note that the system configuration of the medical information processing apparatus 101 according to this embodiment is the same as in the first embodiment described with reference to FIG. 1, and the hardware configuration is the same as in the first embodiment described with reference to FIG. 2, and therefore, a description thereof will be omitted.

(Functional Blocks)

The functional blocks of the medical information processing apparatus 101 according to the second embodiment are the same as in the first embodiment described with reference to FIG. 3. In the first embodiment, the finding selection unit 302 shown in FIG. 3 performs processing of estimating a diagnostic name from imaging findings, calculating the influence degree of each imaging finding for the estimated diagnostic name, and selecting an imaging finding whose influence degree calculated is a predetermined value or more as an imaging finding to be used to generate a report. In the second embodiment, a finding selection unit 302 selects an imaging finding to be used to generate a report not only based on the influence degree for diagnostic name inference but also based on an instruction from the user via a user interface screen (not shown).

A selection information storage unit 304 stores, in to finding selection information table 314, selection information (specification information) that is information for specifying the imaging finding selected by the finding selection unit 302. In this embodiment, the selection information storage unit 304 stores information for specifying whether selection is based on an instruction from the user in the finding selection information table 314 in addition to the information for specifying an imaging finding used for a report.

In addition to the type and value of an imaging finding and a designation concerning whether the finding is a finding described in a report or a finding that is not described in a report, a search condition obtaining unit 305 obtains, as search condition information, a designation representing that the finding is a finding described in a report based on an instruction from the user.

In accordance with the search condition information obtained by the search condition obtaining unit 305, a search unit 306 searches for a similar case while discriminating whether an imaging finding is described in a report based on an instruction from the user in addition to the type and value of the imaging finding and a designation concerning whether the imaging finding is described in a report or not.

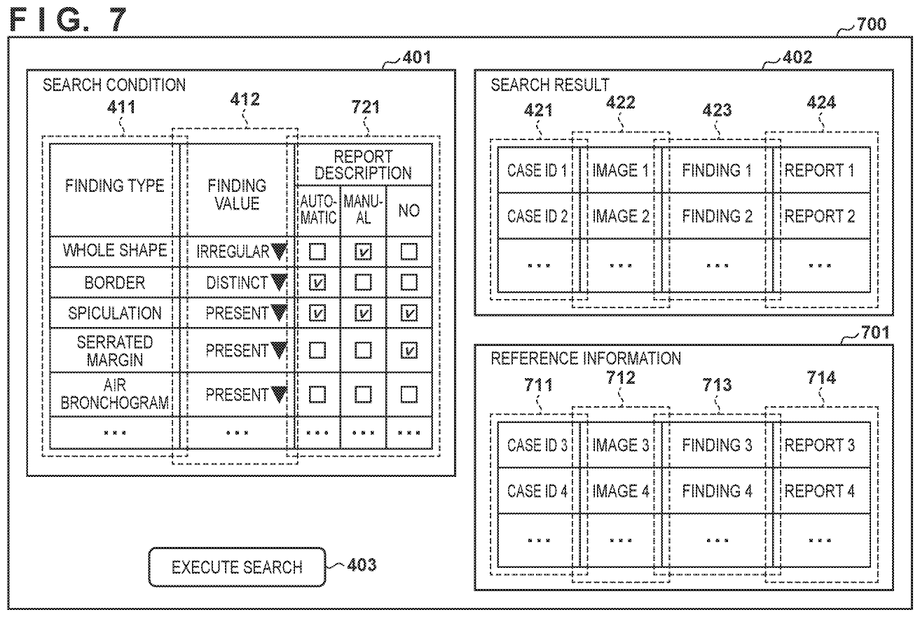

A search result display unit 307 displays the search result by the search unit 306 together with the search condition information obtained by the search condition obtaining unit 305. Also, in addition to the search condition information and the search result, the search result display unit 307 displays a case which has an imaging finding of a value designated by the user but whose condition concerning the description in a report does not correspond to the search condition on the user interface screen as reference information. The user interface screen will be described with reference to FIG. 7.

(User Interface Screen)

FIG. 7 is a view showing an example of a user interface screen 700 of the medical information processing apparatus 101 according to the second embodiment. In the user interface screen (FIG. 4) according to the first embodiment, each row of the report description designation region 413 is provided with two check boxes configured to designate whether the imaging finding of each finding type displayed in the finding type display region 411 is a finding described in a report or a finding that is not described in a report.

In the user interface screen 700 according to the second embodiment, the report description designation region 413 according to the first embodiment is changed to a report description designation region 721 capable of designating whether a finding is described in a report based on an instruction from the user. A reference information display region 701 is also added to the user interface screen 700 according to the second embodiment.

The report description designation region 721 is configured to designate whether a designated imaging finding is a finding automatically selected based on an influence degree and described in a report, a finding selected based on an instruction from the user and described in a report, or a finding that is not described in a report. That is, each row of the report description designation region 721 is provided with three check boxes such that a search condition can be set while discriminating a finding automatically described in a report (“automatic”), a finding manually described in a report based on an instruction from the user (“manual”), and a finding that is not described in a report (“absent”).

When a check box in a column “automatic” is checked, a case whose finding is automatically selected and described in a report is searched by the search unit 306. When a check box in a column “manual” is checked, a case whose finding is selected based on an instruction from the user and described in a report is searched by the search unit 306. When a check box in a column “absent” is checked, a case whose finding is not described in a report is searched by the search unit 306.

In the example shown in FIG. 7, cases in which “whole shape” is “irregular” (described in a report based on a user instruction), “border” is “distinct” (automatically described in a report), “spiculation” is “present” (a description in a report does not matter), and “serrated margin” is “present” (not described in a report) are searched.

The search result display unit 307 displays a case which has an imaging finding of a value designated as a search condition but whose condition concerning the description in a report does not correspond to the search condition in the reference information display region 701 as reference information. In the reference information display region 701, a region 711 is a region configured to display the case ID of the case, and a region 712 is a region configured to display a medical image of the case. In addition, a region 713 is a region configured to display an imaging finding of the case, and a region 714 is a region configured to display a report of the case.

(Report Generation Processing Procedure)

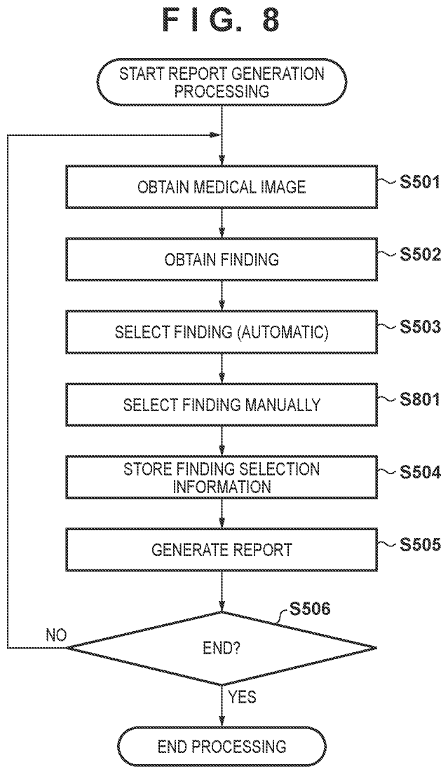

FIG. 8 is a flowchart showing the procedure of report generation processing of the medical information processing apparatus 101 according to this embodiment. In the processing procedure according to this embodiment, step S801 is added to the processing procedure described in the first embodiment with reference to FIG. 5.

In step S801, the finding selection unit 302 selects imaging findings to be used to generate a report based on an instruction from the user via a user interface screen (not shown). In this embodiment, the finding selection unit 302 and the user interface screen function as a second finding selection unit configured to select a finding to be used to generate a report based on an instruction from the user.

In step S504, based on the imaging findings selected in step S503, the selection information storage unit 304 stores, in the finding selection information table 314, finding selection information for specifying a finding described in a report (an imaging finding used to generate a report). Also, in this embodiment, the selection information storage unit 304 stores, in the finding selection information table 314, information (second specification information) for discriminating the imaging findings selected based on the instruction from the user in step S801 and the imaging findings selected in step S503. The selection information storage unit 304 stores, in the finding selection information table 314, the second specification information for discriminating the findings selected by the finding selection unit 302 (first finding selection unit) and the findings selected by the finding selection unit 302 and the user interface screen (second finding selection unit).

(Search Processing Procedure)

The search processing procedure of the medical information processing apparatus 101 according to this embodiment is the same as in the first embodiment described with reference to FIG. 6.

In step S611, the search condition obtaining unit 305 obtains search condition information via a search condition designation region 401 (FIG. 7). In this embodiment, the search condition obtaining unit 305 also obtains a designation concerning whether a finding is a finding described in a report based on an instruction of the user in the report description designation region 721. That is, the search condition obtaining unit 305 obtains, via the search condition designation region 401, search conditions including a designated finding designated as a condition to search for a similar case of a case and second discrimination information (information designated in the report description designation region 721) for discriminating whether the designated finding is a finding selected by the finding selection unit 302 (first finding selection unit), a finding selected by the finding selection unit 302 and the user interface screen (second finding selection unit), or a finding that is not described in a report.

In step S612, in accordance with the search condition information obtained in step S611, the search unit 306 performs a search while discriminating a case whose finding designated as search condition information is described in a report based on a user instruction in addition to the type and value of an imaging finding and a designation concerning whether the imaging finding is described in a report or not. The search unit 306 searches for a similar case based on the second specification information in step S504 and the second discrimination information in step S611 while discriminating whether the designated finding is a finding selected by the finding selection unit 302 (first finding selection unit), a finding selected by the finding selection unit 302 and the user interface screen (second finding selection unit), or a finding that is not described in a report.

In step S613, the search result display unit 307 displays the result of the search in step S612 in a search result display region 402. Also, the search result display unit 307 displays a case which has an imaging finding of a value designated as a search condition but whose condition concerning the description in a report does not correspond to the search condition in the reference information display region 701 as reference information.

The search result display unit 307 displays the search result while discriminating whether each of findings (finding 1, finding 2, finding 3, . . . ) in a finding display region 423, which are searched based on designated findings, is a finding selected by the finding selection unit 302 (first finding selection unit), a finding selected by the finding selection unit 302 and the user interface screen (second finding selection unit), or a finding that is not described in a report.

The search result display unit 307 displays a case that matches the value of the designated finding but does not correspond to the condition of the second discrimination information in the search result of the search unit 306 in the reference information display region 701 as reference information.

As described above, according to this embodiment, when searching for a case based on an imaging finding, search processing is executed by designating an imaging finding as a search condition and also designating whether the imaging finding is automatically described in a report, described in a report based on a user instruction, or not described. This makes it possible to search for a case while discriminating an imaging finding automatically described in a report, an imaging finding manually described in a report based on a user instruction, and an imaging finding that is not described.

### Modification of Second Embodiment

The finding selection unit 302 may select, based on a user instruction by voice input, an imaging finding to be used for generate a report from a plurality of imaging findings obtained by a finding obtaining unit 301. The imaging finding to be used to generate a report may be designated via another apparatus included in a medical information processing system 10 or another system. The imaging finding to be used to generate a report may be designated via setting data set in advance. The finding selection unit 302 may select an imaging finding that matches a condition defined by the user from the plurality of imaging findings obtained by the finding obtaining unit 301.

The selection information storage unit 304 may store information for specifying an imaging finding used to described a report based on a user instruction as an attribute item of a finding table 312 in addition to the imaging finding selected by the finding selection unit 302. In addition to the finding table 312, a new table may be defined to store the information for specifying an imaging finding used to described a report based on a user instruction. The information may be stored in a database or a storage area different from the case DB 102. Also, the search result display unit 307 may control display in the search result display region 402 by displaying each imaging finding in a different mode (for example, a mode discriminated by changing at least one of the display color and the display size) based on the presence/absence of a description in a report based on a user instruction.

According to the embodiments of the present invention, a case can be searched while discriminating whether a finding designated as a search condition is a finding described in a report or a finding that is not described in a report.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2020-107226, filed Jun. 22, 2020, which is hereby incorporated by reference herein in its entirety.

What is claimed is:

1. A medical information processing apparatus comprising:
a memory storing a program; and
one or more processors which, by executing the program, cause the information processing apparatus to:
obtain a plurality of findings for a case from a medical image as a target of a diagnosis;
generate a description of a report based on at least some of the plurality of findings; and
search a storage unit storing a plurality of cases for a specific case using a search condition including a designated finding designated as a condition to search for the specific case and discrimination information for discriminating whether the designated finding is a finding described in the report while discriminating whether the designated finding is a finding described in the report or a finding that is not described in the report.

2. The apparatus according to claim 1, wherein the storage unit stores specification information for specifying the finding used to describe the report for every plurality of cases, and the information processing apparatus searches for the specific case based on the specification information and the discrimination information.

3. The apparatus according to claim 2, wherein the one or more processors which, by executing the program, further cause the information processing apparatus to:
store, in the storage unit, the specification information for specifying the finding used to describe the report;
obtain the search condition including the designated finding designated as the condition to search for the specific case and the discrimination information for discriminating whether the designated finding is the finding described in the report; and
display a search result by the information processing apparatus on a display unit together with the search condition,
wherein the information processing apparatus displays the search result on the display unit while discriminating whether the finding searched based on the designated finding is a finding described in the report or a finding that is not described in the report.

4. The apparatus according to claim 3, wherein the one or more processors which, by executing the program, further cause the information processing apparatus to:
select, from the plurality of findings, at least some findings as findings to be used to generate the report; and
select the findings to be used to generate the report based on an instruction from a user.

5. The apparatus according to claim 4, wherein the information processing apparatus stores, in the storage unit, second specification information for discriminating a finding selected by the information processing apparatus and a finding selected by the information processing apparatus.

6. The apparatus according to claim 5, wherein the information processing apparatus obtains the search condition including
the designated finding, and
second discrimination information for discriminating whether the designated finding is a finding selected by the information processing apparatus, a finding selected by the information processing apparatus, or a finding that is not described in the report.

7. The apparatus according to claim 7, wherein the information processing apparatus searches for the specific case while discriminating, based on the second specification information and the second discrimination information, whether the designated finding is a finding selected by the information processing apparatus, a finding selected by the information processing apparatus, or a finding that is not described in the report.

8. The apparatus according to claim 4, wherein the information processing apparatus displays the search result on the display unit while discriminating whether the finding searched based on the designated finding is a finding selected by the information processing apparatus, a finding selected by the information processing apparatus, or a finding that is not described in the report.

9. The apparatus according to claim 3, wherein the information processing apparatus displays the finding on the display unit discriminatively by changing at least one of a display color and a display size.

10. The apparatus according to claim 3, wherein the information processing apparatus displays, on the display unit, information for identifying the specific case searched by the information processing apparatus and a medical image representing the specific case.

11. The apparatus according to claim 6, wherein the information processing apparatus displays a case that matches a value of the designated finding but does not correspond to a condition of the second discrimination information in the search result of the information processing apparatus on the display unit as reference information.

12. The apparatus according to claim 4, wherein the information processing apparatus calculates an influence degree of each finding for a diagnostic name estimated from the plurality of findings, and selects an imaging finding whose influence degree is not less than a predetermined value.

13. The apparatus according to claim 1, wherein the information processing apparatus generates the description of the report by applying the finding to a template set in advance.

14. The apparatus according to claim 1, wherein the information processing apparatus obtains the medical image as the target of the diagnosis and analyzes the medical image, thereby obtaining the plurality of findings.

15. A medical information processing method comprising:
obtaining a plurality of findings for a case from a medical image as a target of a diagnosis;
generating a description of a report based on at least some of the plurality of findings; and
searching, a storage unit storing a plurality of cases for a specific case using a search condition including a designated finding designated as a condition to search for the specific case and discrimination information for discriminating whether the designated finding is a finding described in the report while discriminating whether the designated finding is a finding described in the report or a finding that is not described in the report.

16. A non-transitory computer-readable medium storing a program for causing a computer to execute the medical information processing method according to claim 15.
